(19) United States
(12) Patent Application Publication
(10) Pub. No.: US 2016/0220497 A1
(43) Pub. Date: Aug. 4, 2016

(54) Method For The Therapeutic Treatment Of Keratinous Substrate, Mucous Membrane Or Tooth

(71) Applicant: Dow Corning Corporation, Midland, MI (US)

(72) Inventors: Virginie Caprasse, Oreye (BE); Andre Colas, Rixensart (BE); Fabrizio Galeone, Epinois (BE); Arnaud Labrosse, Obaix (BE); Leon Marteaux, Auderghem (BE); Anke Sieg, Bruxelles (BE); Xavier Thomas, Famars (FR)

(21) Appl. No.: 14/410,576

(22) PCT Filed: Jun. 17, 2013
(86) PCT No.: PCT/EP2013/062541

§ 371 Date: Dec. 23, 2014

(30) Foreign Application Priority Data
Jun. 25, 2012 (EP) 12305737.4
Jun. 26, 2012 (EP) 12305749.9

(51) Int. Cl. A61K 9/50; A61K 47/02; A61K 9/70; A61K 47/34; A61K 47/24

(52) U.S. Cl. CPC A61K 9/50; A61K 47/34; A61K 47/24; A61K 9/7015; A61K 47/02; A61K 9/501

(57) ABSTRACT

The invention relates to a method for the therapeutic treatment of keratinous substrate, mucous membrane or tooth comprising preparing a composition containing water, at least one therapeutically active ingredient, an organopolysiloxane X having at least two silicon-bonded alkenyl groups per molecule, a hydrosilylation catalyst and an organohydrogensiloxane compound Y having at least two silicon bonded hydrogen atoms per molecule. At least one of compound X or Y is enclosed within microcapsules suspended in an aqueous phase, and the composition is applied in a galenic form preferably chosen from spray, foam, brush, pen or roller so that a hydrosilylation reaction between compounds X and Y occurs to form a film on the keratinous substrate, mucous membrane or tooth. The invention further extends to a delivery system and to a film comprising a cured silicone.

The present disclosure generally relates to a method for the therapeutic treatment of keratinous substrate, mucous membrane or tooth comprising preparing a composition containing water, at least one therapeutically active ingredient, an organopolysiloxane X having at least two silicon-bonded alkenyl groups per molecule, a hydrosilylation catalyst and an organohydrogensiloxane compound Y having at least two silicon bonded hydrogen atoms per molecule.

Gel compositions are known that can be used to deliver drug or other active ingredients, wherein the gel is formed from hydrosilylation reaction of compounds X and Y in the presence of a hydrosilylation catalyst. The gel composition further contains an active ingredient. Such a gel is for example disclosed in US2010/0183525. However these gel compositions lack stability in time and should be used as they are formed without long storage capabilities. Accordingly, there remains an opportunity to develop improved methods for the therapeutic treatment of keratinous substrate, mucous membrane or tooth. Keratinous substrates include skin lips teguments and hair. Mucous membrane are for example buccal or nasal membranes.

The present invention provides a method for the therapeutic treatment of keratinous substrate, mucous membrane or tooth comprising preparing a composition containing water, at least one therapeutically active ingredient, an organopolysiloxane X having at least two silicon-bonded alkenyl groups per molecule, a hydrosilylation catalyst and an organohydrogensiloxane compound Y having at least two silicon bonded hydrogen atoms per molecule wherein at least one of compound X or Y is enclosed within microcapsules suspended in an aqueous phase, and the composition is applied in a galenic form—preferably chosen from spray, foam, brush, pen or roller—so that a hydrosilylation reaction between compounds X and Y occurs to form a film on the keratinous substrate, mucous membrane or tooth. The invention also provides a method for manufacturing a medicament intended for therapeutic treatment of keratinous substrate, mucous membrane or tooth comprising preparing a composition containing water, at least one therapeutically active ingredient, an organopolysiloxane X having at least two silicon-bonded alkenyl groups per molecule, a hydrosilylation catalyst and an organohydrogensiloxane compound Y having at least two silicon bonded hydrogen atoms per molecule wherein at least one of compound X or Y is enclosed within microcapsules suspended in an aqueous phase, and the composition is applied in a galenic form so that a hydrosilylation reaction between compounds X and Y occurs to form a film on the keratinous substrate, mucous membrane or tooth.

The invention further provides a delivery system for the therapeutic treatment of keratinous substrate, mucous membrane or tooth comprising a composition containing water, at least one therapeutically active ingredient, an organopolysiloxane X having at least two silicon-bonded alkenyl groups per molecule, a hydrosilylation catalyst and an organohydrogensiloxane compound Y having at least two silicon bonded hydrogen atoms per molecule wherein at least one of compound X or Y is enclosed within microcapsules suspended in an aqueous phase, and the composition is applied in a galenic form, preferably chosen from spray, foam, brush, pen or roller, so that a hydrosilylation reaction between compounds X and Y occurs to form a film on the keratinous substrate, mucous membrane or tooth.

The invention also provides a film comprising a composition containing water, at least one therapeutically active ingredient, a cured organopolysiloxane formed from the reaction of an organopolysiloxane X having at least two silicon-bonded alkenyl groups per molecule, and an organohydrogensiloxane compound Y having at least two silicon bonded hydrogen atoms per molecule in the presence of a hydrosilylation catalyst wherein at least one of compound X or Y is enclosed within microcapsules suspended in an aqueous phase, and the composition containing compounds X and Y is applied in a galenic form so that a hydrosilylation reaction between compounds X and Y occurs to form a film on the keratinous substrate, mucous membrane or tooth.

Galenic formulation deals with the principles of preparing and compounding medicines in order to optimize their absorption. Today, galenic formulation is part of pharmaceutical formulation. The pharmaceutical formulation of a medicine has an impact on the pharmacokinetics, pharmacodynamics and safety profile of a drug. A galenic form designates the individual form under which are placed the active ingredients and the excipients (inactive materials) to constitute a drug. It corresponds to the final physical aspect of the drug such as it will be used by a patient. Preferably the galenic form is chosen from spray, foam, brush, pen or roller. These forms are well adapted to therapeutically treat keratinous substrate, mucous membrane or tooth. The film is preferably adhesive and/or coherent i.e. it stays together as a continuous film on the surface to be treated.

The present invention provides compositions having improved storage stability compared to many gel based compositions. In drug applications, extended periods of time of storage stability are desired. For example up to 6 months or over 3 years storage stability is desired. The delivery system according to the present invention can be stable for these extended periods and even more. The presence of at least one of the component X or Y in a microcapsule permits to delay the curing reaction until time of application of the composition on keratinous substrate, mucous membrane or tooth. Moreover, the application in the form of a spray, roller brush, pen or foam enables to treat surfaces affected for example by skin diseases which are difficult to access body places.

The method and delivery system provides one part room temperature curable suspensions using methods of application that do not require further application by hand like spray foam brush pen roller and the like.

The system can be delivered in the form of a spray, such as a pump spray or an aerosol spray or it can be delivered as a foam, brush, pen or by a roller. Preferably it is delivered as a spray. This is a particularly appropriate way to treat damaged surfaces to be healed which can be used to treat difficult to access body places.

The delivery of the composition as a film on the surface to be treated is enabled by the chosen format i.e. spray foam or roller and by the choice of at least one of the reacting components present in a microcapsule which protects from premature reaction. The inventors believe that some reaction may occur in the microcapsules during storage which forms partially polymerized components which may further crosslink when the microcapsules burst during application of the composition to the surface to be treated.

The compositions may be stable over time up to the temperature of around 45° C. and remain effective for forming a silicone polymer film adhering to the surfaces to be treated. When the compositions are spread in the form of a film over a support, for example a keratinous material, the microcapsules break up under the pressure of application and also under the effect of dehydration of the film deposited and the compounds X and Y then brought into intimate contact in the presence of the catalyst react together to form a film.

Preferably, the catalyst is present in said encapsulated compound X or compound Y, the microcapsules being in suspension in aqueous phase.

Preferably, compounds X and Y are present in separate encapsulated forms.

This is particularly appropriate to ensure that the final crosslinking reaction only takes place at the time of application.

In a preferred embodiment, the composition contains a first population of microcapsules containing compound X and a second population of microcapsules containing compound Y, the catalyst being present in one of the populations of microcapsules preferably the one containing compound X.

Preferably, a first portion of the microcapsules comprise the compound X and the catalyst and a second portion comprises the compound Y, optionally associated with the compound X.

Preferably, the molar portion of SiH/unsaturated groups ranges from 1 to 20, more preferably from 3 to 10. These ratios tend to promote curing as thin films and provide adequate storage stability in dispersions.

In a preferred embodiment, the organopolysiloxane X comprises at least two siloxane units per molecule that each independently have the average formula R2RmSiO(3-m)/2, wherein each R is independently a hydrocarbon group having from 1 to 20 carbon atoms, each R2 is a monovalent alkenyl aliphatic group, and m is a number of from 0 to 2.

Preferably, the organopolysiloxane X comprises units of average formula R2R2SiO1/2, R2RSiO and/or R2SiO3/2 wherein each R is independently a hydrocarbon group having from 1 to 20 carbon atoms and each R2 is a monovalent alkenyl aliphatic group.

Preferably, the organopolysiloxane X has an average formula that is defined as:

CH₂═CH(Me)₂SiO[Me₂SiO]_{x′}Si(Me)₂CH═CH₂;

CH₂═CH—(CH₂)₄—(Me)₂SiO[Me₂SiO]_{x′}Si(Me)₂—(CH₂)₄—CH═CH₂;

or

Me₃SiO[(Me)₂SiO]_{x′}[CH₂═CH(Me)SiO]_{x″}SiMe₃,

and wherein Me is methyl, x′≧0, and x″≧2.

In a preferred embodiment, the organohydrogensiloxane compound Y has an average formula (R33SiO1/2)a(R42SiO2/2)b(R4HSiO2/2)c, wherein:

each R³ is independently a hydrogen atom or R⁴,
each R⁴ is independently a monovalent hydrocarbyl having 1 to 10 carbon atoms, and wherein
a≧2, b≧0, and c≧2.

Preferably, the organohydrogensiloxane Y is a dimethyl, methyl-hydrogen polysiloxane having an average formula (CH3)3SiO[(CH3)2SiO]b[(CH3)HSiO]cSi(CH3)3, wherein b≧0, and c≧2.

According to a preferred embodiment of the invention, the hydrosilylation catalyst is a platinum group metal present at a concentration of 1 to 500 parts per million relative to the total weight of film.

Preferably, the composition contains from 0.001 to 20 weight percent therapeutically active ingredient based on the total weight of the dispersion.

Preferably, the microcapsule shell comprises silica.

Preferably, the shell of the microcapsules are formed from precursors comprising tetraalkoxysilane.

Preferably, the microcapsules have a mean average particle size according to D(v,0.5) of less than 20 micrometers.

While not intending to be bound by any particular theory, it is believed that the amounts of the organopolysiloxane X and the organohydrogensiloxane Y promote partial reactions and promote more efficient overall curing. It is theorized that small amounts of the hydrosilylation catalyst may permeate through the encapsulated particles of the second population and catalyze some reactions between the organopolysiloxane X and the organohydrogensiloxane Y.

The dispersion includes water and a plurality of encapsulated particles dispersed in the water. If the encapsulated particles are described as solids dispersed in the water, then the dispersion may be further defined as a sol, suspension, gel, or colloidal solution. Colloidal solutions tend to include particles of less than 100 nanometers in size dispersed in the continuous phase. If the encapsulated particles are described as liquids, then the dispersion may be further defined as an emulsion such as an oil in water (O/W) emulsion, water in oil (W/O) emulsion, water in oil in water (W/O/W) emulsion, ionic or nonionic emulsion, anionic, cationic, or amphoteric emulsion, microemulsion, miniemulsion, multiple emulsion, artificial emulsion, and the like.

The water of the dispersion may be tap water, well water, purified water, deionized water, and combinations thereof and may be present in the dispersion in varying amounts depending on the type of dispersion. The water may be the continuous phase and the plurality of encapsulated particles may be the dispersed phase. In various embodiments, the water is present in amounts of from 1 to 99, of from 5 to 95, 10 to 90, 15 to 85, 20 to 80, 25 to 75, 30 to 70, 35 to 65, 40 to 60, 45 to 55, from 5 to 70, from 10 to 70, from 20 to 70, from 30 to 70, from 40 to 70, from 50 to 70, or from 60 to 70, or about 50, parts by weight per 100 parts by weight of the dispersion. Alternatively, the water is present as a balance of the dispersion that includes the plurality of encapsulated particles and the active agent.

It is also contemplated that one or more supplementary solvents may be combined with the water. The supplemental solvents may be hydrophilic and polar and may include alcohols, solvents that include —OH groups, ethers, esters, and the like. Further, the water may be combined with one or more drug delivery enhancers (such as propylene glycol and pentylene glycol), occlusive agents (such as petrolatum and mineral oil), or any of the additives, surfactants, or other components described in greater detail below.

### Organopolysiloxane X—At Least Two Silicon-Bonded Alkenyl Groups Per Molecule

Organopolysiloxanes are polymers including siloxy units independently selected from (R₃SiO_{1/2}), (R₂SiO_{2/2}), (RSiO_{3/2}), or (SiO_{4/2}) siloxy units, where R may be a hydrocarbon group. These siloxy units can be combined in various manners to form cyclic, linear, or branched structures. The chemical and physical properties of the resulting polymeric structures can vary. For example organopolysiloxanes can be volatile fluids, low viscosity fluids, high viscosity fluids/gums, elastomers, rubbers, or resins.

The (a) first organopolysiloxane having at least two silicon-bonded alkenyl groups per molecule may be selected from any organopolysiloxane, or mixture of organopolysiloxanes including at least two siloxy units represented by the formula R²RₘSiO_{(3-m)/2} wherein R is independently a hydrocarbon group having from 1 to 20 carbon atoms, each R² is a monovalent alkenyl group, e.g. having from 2 to 12 carbon atoms, and m is a number of from 0 to 2. The R² alkenyl groups of the (a) first organopolysiloxane having at least two silicon-bonded alkenyl groups per molecule are exemplified by vinyl, allyl, 3-butenyl, 4-pentenyl, 5-hexenyl, 6-heptenyl, 7-octenyl, 8-nonenyl, 9-decenyl, 10-undecenyl, 4,7-octadienyl, 5,8-nonadienyl, 5,9-decadienyl, 6,11-dodecadienyl and 4,8-nonadienyl. The R² alkenyl group may be present on any mono, di, or tri siloxy unit in the organopolysiloxane, for example, (R²R₂SiO_{1/2}), (R²RSiO_{2/2}), or (R²SiO_{3/2}), as well as in combination with other siloxy units not including an R² substituent, such as (R₃SiO_{1/2}), (R₂SiO_{2/2}), (RSiO_{3/2}), or (SiO_{4/2}) siloxy units where R is a hydrocarbon including 1 to 20 carbons, alternatively an alkyl group including 1 to 12 carbons, alternatively an alkyl group including 1 to 6 carbons or alternatively methyl providing there are at least two R² substituents in the organopolysiloxane. The monovalent hydrocarbon group R having from 1 to 20 carbon atoms is exemplified by alkyl groups such as methyl, ethyl, propyl, butyl, hexyl, octyl, and decyl, cycloaliphatic groups such as cyclohexyl, aryl groups such as phenyl, tolyl, and xylyl, and aralkyl groups such as benzyl and phenylethyl.

Representative, non-limiting, examples of such organopolysiloxanes suitable as the (a) first organopolysiloxane having at least two silicon-bonded alkenyl groups per molecule include those having the average formula (R₂R²SiO_{1/2})ᵥ(R₂SiO_{2/2})ₓ, (R₂R²SiO_{1/2})ᵥ(R₂SiO_{2/2})ₓ(R²RSiO_{2/2})_{y}, (R₂R²SiO_{1/2})ᵥ(R₂SiO_{2/2})ₓ(RSiO_{3/2})_{z}, (R₂R²SiO_{1/2})ᵥ(R₂SiO_{2/2})ₓ(RSiO_{3/2})_{z}(SiO_{4/2})_{w}, (R₂R²SiO_{1/2})ᵥ(SiO₂)_{w}(R₂SiO)ₓ, (R₃SiO_{1/2})ᵥ(R₂SiO)ₓ(R²RSiO_{2/2})_{y}, (R₃SiO_{1/2})ᵥ(R₂SiO)ₓ(R²RSiO)_{y}, (R₃SiO_{1/2})ᵥ(R₂SiO)ₓ(R²RSiO)_{y}(RSiO_{3/2})_{z}, (R₃SiO_{1/2})ᵥ(R₂SiO)ₓ(R²RSiO)_{y}(SiO₂)_{w}, (R₃SiO_{1/2})ᵥ(R₂SiO)ₓ (R²RSiO)_{y}(SiO₂)_{w}(RSiO_{3/2})_{z}, and/or (R₃SiO_{1/2})ᵥ(R₂SiO)ₓ(R²SiO_{3/2})_{z}, where v≧2, w≧0, x≧0, y≧2, and z is ≧0, and wherein R and R² are as described above.

The (a) first organopolysiloxane may also be or include a mixture of any of the aforementioned organopolysiloxanes. The molecular weight of the (a) first organopolysiloxane may vary, and is not limiting. However, when molecular weights become too high, or if the (a) first organopolysiloxane is a solid, it may be difficult to handle or incorporate the (a) first organopolysiloxane in the encapsulated particles. Thus, it may be desirable to dilute the (a) first organopolysiloxane in a suitable solvent or lower molecular weight fluid, such as a less viscous silicone fluid. The viscosity of the (a) first organopolysiloxane or dispersion of the (a) first organopolysiloxane in the lower molecular weight fluid may vary from 1 to 10,000 mPa·s, alternatively, 50 to 1000 mPa·s , or alternatively, 100 to 1000 mPa·s, when measured at 25° C.

In various embodiments, the (a) first organopolysiloxane is selected from the group consisting of trimethylsiloxy-terminated polydimethylsiloxane-polymethylvinylsiloxane copolymers, vinyldimethylsiloxy-terminated polydimethylsiloxane-polymethylvinylsiloxane copolymers, trimethylsiloxy-terminated polydimethylsiloxane-polymethylhexenylsiloxane copolymers, hexenyldimethylsiloxy-terminated polydimethylsiloxane-polymethylhexenyl siloxane copolymers, trimethylsiloxy-terminated polymethylvinylsiloxane polymers, trimethylsiloxy-terminated polymethylhexenylsiloxane polymers, vinyldimethylsiloxy-terminated polydimethylsiloxane polymers, and hexenyldimethylsiloxy-terminated polydimethylsiloxane polymers, each having a degree of polymerization of from 10 to 300, or alternatively having a viscosity at 25° C. of 10 to 1000 mPa·s.

Alternatively the (a) first organopolysiloxane may be selected from vinyl functional endblocked polydimethylsiloxanes (vinyl siloxanes) or hexenyl functional endblocked polydimethylsiloxanes (hexenyl siloxanes), such as those having the average formula CH₂═CH(Me)₂SiO[Me₂SiO]_{x′}Si(Me)₂CH═CH₂, CH₂═CH—(CH₂)₄—(Me)₂SiO[Me₂SiO]_{x′}Si(Me)₂—(CH₂)₄—CH═CH₂, or Me₃SiO[(Me)₂SiO]_{x′}[CH₂═CH(Me)SiO]_{x″}SiMe₃, wherein Me is methyl, x′≧0, alternatively x is 0 to 200, alternatively x is 10 to 150, x″≧2, alternatively x″ is 2 to 50, and alternatively x″ is 2 to 10.

Vinyl or hexenyl functional polydimethylsiloxanes may be used and non-limiting examples include DOW CORNING® fluids, SFD 128, DC4-2764, DC2-7891, DC2-7754, DC2-7891, and DC 2-7463, SFD-117, SFD-119, SFD 120, SFD 129, DC 5-8709, LV, 2-7038, DC 2-7892, 2-7287, 2-7463, and dihexenyl terminal DC7692, DC7697 (Dow Corning Corporation, Midland, Mich.).

### Hydrosilylation Catalyst

The hydrosilylation catalyst may be any suitable Group VIII metal compound based catalyst selected from a platinum, rhodium, iridium, palladium, and/or ruthenium. Group VIII group metal including catalysts useful in this disclosure can be any of those known to catalyze reactions of silicon bonded hydrogen atoms with silicon bonded unsaturated hydrocarbon groups, e.g. in hydrosilylation reaction. The preferred Group VIII metal for use in this disclosure is a platinum based catalyst. Some preferred platinum based catalysts include, but are not limited to, platinum metal, platinum compounds and platinum complexes.

Non-limiting examples of suitable (b) hydrosilylation catalysts are described in U.S. Pat. No. 2,823,218 (commonly referred to as “Speier's catalyst) and U.S. Pat. No. 3,923,705, expressly incorporated herein by reference. The (b) hydrosilylation catalyst may be a “Karstedt's catalyst”, which is described in U.S. Pat. Nos. 3,715,334 and 3,814,730, expressly incorporated herein by reference. Karstedt's catalyst is a platinum divinyl tetramethyl disiloxane complex typically including about one-weight percent of platinum in a solvent such as toluene. Alternatively the (b) hydrosilylation catalyst may include or be a reaction product of chloroplatinic acid and an organosilicon compound including terminal aliphatic unsaturation, as described in U.S. Pat. No. 3,419,593, expressly incorporated herein by reference. Alternatively, the (b) hydrosilylation catalyst may include a neutralized complex of platinum chloride and divinyl tetramethyl disiloxane, as described in U.S. Pat. No. 5,175,325, also incorporated herein by reference.

Preferably, the hydrosilylation catalyst is a platinum group metal present at a concentration of 1 to 500 parts per million relative to the total weight of film after evaporation of water.

### Organohydrogensiloxane Y

Organohydrogensiloxanes are organopolysiloxanes having at least one SiH including siloxy unit, that is at least one siloxy unit in the organopolysiloxane has the formula (R₂HSiO_{1/2}), (RHSiO_{2/2}), or (HSiO_{3/2}). The organohydrogensiloxane Y having at least two silicon bonded hydrogen atoms per molecule is not particularly limited and may include any organopolysiloxane including a silicon-bonded hydrogen atom (SiH). Thus, the organohydrogensiloxane may include any number of (R₃SiO_{1/2}), (R₂SiO_{2/2}), (RSiO_{3/2}), (R₂HSiO_{1/2}), (RHSiO_{2/2}), (HSiO_{3/2}) or (SiO_{4/2}) siloxy units, providing there are on average at least two SiH siloxy units in the molecule. The organohydrogensiloxane can include or be a single linear or branched organohydrogensiloxane or a combination including two or more linear or branched organohydrogensiloxanes that differ in at least one of structure, viscosity, average molecular weight, siloxane units, and/or sequence. Although not particularly limited, the viscosity of the (c) organohydrogensiloxane is may be of from 3 to 10,000 mPa·s, alternatively from 3 to 1,000 mPa·s, or alternatively from 10 to 500 mPa·s, when measured at 25° C. The organohydrogensiloxane is thus liquid at room temperature.

The amount of SiH units present in the organohydrogensiloxane may vary, providing there are at least two SiH units per molecule. The amount of SiH units present in the organohydrogensiloxane is expressed herein as % SiH which is the weight percent of hydrogen in the organohydrogensiloxane. The % SiH may vary from 0.01 to 10%, alternatively from 0.1 to 5%, or alternatively from 0.5 to 2%.

In various embodiments, the organohydrogensiloxane has the average formula, (R³₃SiO_{1/2})ₐ(R⁴₂SiO_{2/2})_{b}(R⁴HSiO_{2/2})_{c} wherein R³ is hydrogen or R⁴, R⁴ is a monovalent hydrocarbon group having from 1 to 10 carbon atoms, a≧2, b≧0, alternatively b=1 to 500, alternatively b=1 to 200, c≧2, alternatively c=2 to 200, alternatively c=2 to 100. R⁴ may be a substituted or unsubstituted aliphatic or aromatic hydrocarbyl. Monovalent unsubstituted aliphatic hydrocarbyls are exemplified by, but not limited to, alkyl groups such as methyl, ethyl, propyl, pentyl, octyl, undecyl, and octadecyl and cycloalkyl groups such as cyclohexyl. Monovalent substituted aliphatic hydrocarbyls are exemplified by, but not limited to, halogenated alkyl groups such as chloromethyl, 3-chloropropyl, and 3,3,3-trifluoropropyl. The aromatic hydrocarbon group is exemplified by, but not limited to, phenyl, tolyl, xylyl, benzyl, styryl, and 2-phenylethyl.

In other embodiments, the organohydrogensiloxane may include additional siloxy units and have the average formula (R³₃SiO_{1/3})ₐ(R⁴₂SiO_{2/3})_{b}(R⁴HSiO_{2/2})_{c}(R⁴SiO_{3/2})_{d}, (R³₃SiO_{1/2})ₐ(R⁴₂SiO_{2/2})_{b}(R⁴HSiO_{2/2})_{c}(SiO_{4/2})_{d}, (R³₃SiO_{1/2})ₐ(R⁴₂SiO_{2/2})_{b}(R⁴HSiO_{2/2})_{c}(SiO_{4/2})_{d}(R⁴SiO_{3/2})ₑ, or any mixture thereof, where each R³ is independently a hydrogen atom or R⁴, each R⁴ is independently a monovalent hydrocarbyl, e.g. having 1 to 10 carbon atoms, and a≧2, b≧0, c≧2, d≧0, and e is ≧0. In another embodiment, the (c) organohydrogensiloxane is selected from a dimethyl, methyl-hydrogen polysiloxane having the average formula, (CH₃)₃SiO[(CH₃)₂SiO]_{b}[(CH₃)HSiO]_{c}Si(CH₃)₃ where b≧0, alternatively, b=1 to 200, alternatively b=1 to 100, and c≧2, alternatively, c=2 to 100, alternatively c=2 to 50.

### Active Agent

Referring back to the therapeutically active ingredient or active agent, the terminology “active agent” is not particularly limited and may refer to a pharmaceutically active agent, such as a drug, therapeutic agent, etc. The active agent may be hydrophilic or lipophilic and may be further defined as a hydrophilic drug or a lipophilic drug. In one embodiment, the active agent is further defined as a medicine, medication or medicament and may include any chemical substance intended for use in the medical diagnosis, cure, treatment, or prevention of disease. Alternatively, the active agent is further defined as a drug that may be administered transdermally on skin (e.g. mammalian or human skin). However, it is to be appreciated that the active agent is not limited to these applications. In various embodiments, the active agent is chosen from anti-acne agents, antibiotics, antiseptics, antifungals, antibacterials, antimicrobials, biocides, anti-inflammatory, astringents, hormones, anticancer agents, smoking cessation compositions, cardiovasculars, histamine blockers, bronchodilators, analgesics, antiarrythmics, antihistamines, alpha-I blockers, beta blockers, ACE inhibitors, diuretics, antiaggregants, sedatives, tranquillizers, anticonvulsants, anticoagulant agents, vitamins, anti-ageing agents, agents for treating gastric and duodenal ulcers, anticellulites, proteolytic enzymes, healing factors, cell growth nutrients, peptides, antipsoriasis agents, steroids, corticosteroids and others. Specific non-limiting examples of suitable active agents include penicillins, cephalosporins, tetracyclines, macrolides, epinephrine, amphetamines, aspirin, acetominophen, barbiturates, catecholamines, benzodiazepine, thiopental, codeine, morphine, procaine, lidocaine, benzocaine, sulphonamides, ticonazole, perbuterol, furosamide, prazosin, prostaglandins, salbutamol, indomethicane, diclofenac, glafenine, dipyridamole, theophylline and retinol. In one embodiment, the active agent is chosen from the group of coal tar, tazarotene, calcipotriene, calcipotriol, calcipotriol monohydrate, calicineurin inhibitors, betamethasone, etanercept, adalimumab, infliximab, pimecrolimus, clobetasol propionate, glycyrrhetinic acid, zinc pyrithion, miconazole nitrate, zinc oxide, white petrolatum, alitretinoin, liarozole, bimosiamose, hydrocortisone, clobetasol, triamcinolone, fluocinonide, mometasone, desonide, alclometasone, diflorasone, amcinonide, pimecrolimus, tacrolimus, fuorate, metronidazol, tetracycline, calcineurin inhibitors, methotrexate (steroids), cyclosporin (steroid), TNF inhibitors, oral kinase inhibitors, Janus kinase inhibitors, tofacitinib and combinations thereof.

In various embodiments, the active agent is one or more of the following: scopolamine, nitroglycerin, clonidine, estradiol, fentanyl, nicotine, habitrol, testosterone, lidocaine, epinephrine, iontocaine, norethidrone, ethinyl estradiol, norelgestromin, levonorgestrel, oxybutynin, tetracaine, fentanyl HCl, methylphenidate, selegiline, rotigotine, rivastigmine, centella asiatica, retapamulin, alefacept, benzamycin, erythromycin, benzoyl peroxide, botulinum toxin type A, cefazolin, dextrose usp, chlorhexidine gluconate, clindamycin phosphate, pokofilox, desonide, adapalene gel, dynabac, elidel, norethindrone acetate, ketoconazole, azelaic acid, sodium sulfacet amide, terbinafine hydrochloride, betamethasone valerate, butenafine HCl, minoxidil, tacrolimus, becaplermin, tretinoin, ustekinumab, tigecycline, telavancin, levocetirizine dihydrochloride, niacinamide, ibuprofen, acetaminophen, aspirin, silver chloride, panthenol, clotrimazol, vitamin A, vitamin D3, vitamin D3 derivatives, salicylic acid, and/or dexpanthenol. In another embodiment, the active agent is chosen from caffeine, lidocaine, and combinations thereof. It is also contemplated that the active agent may be chosen from lidocaine, niacinamide, ibuprofen, silver chloride, caffeine, and combinations thereof. The active agent may be in the form of solid particles that spread up in and upon the film of crosslinked silicone. For example, it can be an antibacterial agent in the form of Ag, Cu or Au particles.

Preferably, the composition contains from 0.001 to 20 weight percent therapeutically active ingredient based on the total weight of the dispersion.

### Microcapsules

At least one of organopolysiloxane X or Y is enclosed within microcapsules. It is known to encapsulate one of the components of the system in polymer shell microcapsules.

However, certain polymers such as polycaprolactone, polylactides, polyglucolides, polymers of 3-hydroxybutyric acid, vinyl chloride/vinyl acetate copolymers, methacrylic acid/methyl methacrylate copolymers, polyalkylene adipates and polyester polyols prove less advantageous during storage of the composition at temperatures above 40C: indeed, at these high temperatures, and especially during storage over 2 months at 45[deg.]C., the polymer capsules partly lose their sealing ability and a portion of the components of the system escapes from the capsules and can therefore react when they come into contact at the very core of the composition. The composition then no longer exhibits good storage stability properties and under these conditions the system of silicone components begins to react by premature crosslinking within the composition before its use and its application to the keratin materials. Such a composition does not exhibit its best ability to form a film during the contacting of the components during the rupture of the capsules since the reaction is already initiated within the composition. The objective of the present invention is therefore to improve the storage stability of capsules containing the silicone components of the system. The inventors have discovered that the use of capsules having shell comprising silica made it possible to effectively improve the stability of the composition during storage at temperatures in the vicinity of 45° C., without harming the reactivity of its silicone component.

Generally, there are two processes used for preparing microcapsules shells containing silica. The first technique uses an in situ polymerization of a silica precursor (also known as a sol-gel process), after mixing the silica precursor with an oily phase. Representative and non-limiting examples of the in situ polymerization process are described in documents U.S. Pat. No. 6,159,453, U.S. Pat. No. 6,238,650, U.S. Pat. No. 6,3031,49 and WO 2005/009604. The second technique uses an ex situ process in which the polymerization of the silica precursor is carried out via an emulsion polymerization process. Representative and non-limiting examples of this ex situ polymerization process are described in application WO 03/066209. Such ex-situ process permits to form microcapsules with good storage capabilities and is thus preferred. The shell is usually continuous but may be discontinuous at points. The shell includes a silica such as silicon dioxide (SiO₂) (traditionally known as “silica”) or an organo-modified silica (traditionally known as Ormosils) or a silica hybrid. Suitable examples of organo-modified silicas and/or silica hybrids include, but are not limited to, compounds having the general formula [RSi_{1.75}O₃]ₙ or [R₂Si₂O₃]ₙ where R is an organic group and n is an number of at least one. In one embodiment, the silica is formed from a hydrolysis/condensation reaction of tetraalkoxysilane, for example tetraethylorthosilicate (TEOS) and water to form silica (SiO₂) and C₂H₅OH.

In one embodiment, the plurality of encapsulated particles is prepared using a method that includes the following steps:
I) forming an oil phase including the (a) organopolysiloxane, (b) hydrosilylation catalyst, organohydrogensiloxane and combining the oil phase with an aqueous phase (e.g. solution) including a cationic or amphoteric surfactant to form an oil in water emulsion,
II) adding a water-reactive silicon compound to the oil in water emulsion wherein the water-reactive silicon compound includes, for example, a tetraalkoxysilane, and
III) polymerizing the water-reactive silicon compound at an oil/water interface of the oil in water emulsion to form particles of the first and/or second populations including the core and the layer disposed about the core.

The aforementioned embodiments of the method may be utilized once or more than once to form the plurality of encapsulated particles of the first and/or second populations. After formation, the first and second populations of the plurality of encapsulated particles may then combined with each other to form the dispersion of this disclosure.

Relative to Step (I), the oil phase and aqueous solution of the cationic or amphoteric surfactant may be mixed together to form an oil in water emulsion that is different from the dispersion of this disclosure that includes the water and the plurality of encapsulated particles described above. Mixing and emulsion formation may occur using any known techniques in the emulsion art. The oil phase and aqueous solution may be combined using simple stirring techniques. Particle size of the oil in water emulsion may then be reduced before addition of the water-reactive silicon compound by any emulsification device known in the art. Useful emulsification devices include, but are not limited to, homogenizers, sonolators, rotor-stator turbines, colloid mills, microfluidizers, blades, helices, and combination thereof. The particle size of the oil in water emulsion may range from 0.2 to 500 micrometers or from 0.5 micrometers and 100 micrometers.

The weight ratio of the oil phase to the aqueous phase may be between 40:1 and 1:50. Alternatively, the weight ratio of the oil phase to the aqueous phase is between 2:1 and 1:3. A phase inversion process can also be used in which the oil phase is mixed with a surfactant and a small amount of water, for example 2.5 to 10% by weight based on the oil phase, forming a water-in-oil emulsion which inverts to an oil-in-water emulsion upon shearing. Additional water can then be added for dilution. In one embodiment, the density of the oil phase and the density of the aqueous phase are approximately the same, i.e., the densities are “matched”. Alternatively, these densities can be within 2%, 1%, or 0.5% of each other.

Relative to Steps (II) and (III), the water-reactive silicon compound may include one or more alkoxy groups and each alkoxy group may include 1 to 4 carbons and alternatively 1 to 2 carbons. In one embodiment, the water-reactive silicon compound is further defined as a tetralkoxysilane such as tetraethoxysilane (TEOS) which may be utilized in monomeric form or as a liquid partial condensate or oligomer. Alkyl and alkoxy groups of the tetralkoxysilane may include from 1 to 4 carbon atoms or from 1 to 2 carbon atoms. The tetralkoxysilane may hydrolyze and form a network polymer that is a 3-dimensional network of silicone materials around emulsified droplets of one or more of (a), (b), (c), and/or (d).

It is contemplated that the tetraalkoxysilane can be used in conjunction with one or more water-reactive silicon compounds having at least two, alternatively at least 3, Si—OH groups or hydrolysable groups bonded to silicon (e.g. alkoxy or acyloxy groups bonded to silicon). Non-limiting examples of suitable water-reactive silicon compounds include alkyltrialkoxysilanes (e.g. methyltrimethoxysilane) or liquid condensates/oligomers thereof. Examples of suitable hydrolysable groups include alkoxy and acyloxy groups bonded to silicon atoms.

The water-reactive silicon compounds can include 50-100% by weight tetraalkoxysilane and 0-50% trialkoxysilane. Alternatively, the water-reactive silicon compounds may include at least 75% or alternatively 90 to 100% tetraalkoxysilane. In other embodiments, the water-reactive silicon compound includes an alkoxysilane having organofunctional groups such as a quaternized substituted alkyl groups. One typical quaternary alkoxysilane has the formula (CH₃O)₃SiCH₂CH₂CH₂N⁺(CH₃)₂(CH₂)₁₇CH₃Cl⁻.

The water-reactive silicon compound may be added to the oil-in-water emulsion as an undiluted liquid or as a solution in an organic solvent or in an emulsion. The water-reactive silicon compound and the oil-in-water emulsion may be combined or mixed during addition. In various embodiments, the amounts of tetraalkoxysilane in the water-reactive silicon compounds range from 6/1 to 1/13, alternatively from 1.2/1 to 1/7.3, alternatively from 1.3 to 1/6.1, based on the weight of the oil phase of the emulsion.

The tetraalkoxysilane and/or water-reactive silicon compounds may polymerize at the oil/water interface of the emulsion via a condensation reaction which may occur at acidic, neutral or basic pH. The condensation reaction generally occurs at ambient temperature and pressure, but can occur at increased temperature, for example up to 95° C., and increased or decreased pressure, for example under vacuum to strip volatile alcohols produced therein. In various embodiments, step (III) may be further defined as an “ex-situ emulsion polymerization” step wherein a tetraalkoxysilane precursor hydrolyzes and condenses at an oil/water interface leading to the formation of encapsulated particles via phase transfer.

It is contemplated that any catalyst known to promote the polymerization of the water-reactive silicon compound may be added during Step (III) to form the layer disposed about the core. The catalyst may be an oil soluble organic metal compound, for example an organic tin compound, particularly an organotin compound such as a diorganotin diester, for example dimethyl tin di(neodecanoate), dibutyl tin dilaurate or dibutyl tin diacetate, or alternatively a tin carboxylate such as stannous octoate, or an organic titanium compound such as tetrabutyl titanate. An organotin catalyst can, for example, be used at 0.05 to 2% by weight based on the water-reactive silicon compound. An organotin catalyst has the advantage of effective catalysis at neutral pH. The catalyst may be mixed before emulsification to promote condensation of the water-reactive silicon compound at the surface of emulsified droplets. The catalyst can alternatively be utilized before addition of the water-reactive silicon compound, simultaneously with the water-reactive silicon compound, or after the addition of the water-reactive silicon compound to harden and make more impervious the layer. Encapsulation of the core can however be achieved without catalyst. The catalyst, when used, can be added undiluted, or as a solution in an organic solvent such as a hydrocarbon, alcohol or ketone, or as a multi-phase system such as an emulsion or suspension.

In an alternative embodiment, the method includes the steps of:
A. forming a first oil phase comprising the organopolysiloxane X, and hydrosilylation catalyst,
B. combining the oil phase with an aqueous phase comprising a surfactant to form an oil in water emulsion having an oil/water interface,
C. adding a tetraalkoxysilane to the oil in water emulsion,
D. polymerizing the tetraalkoxysilane at the oil/water interface of the emulsion to form the first population of encapsulated particles wherein the layer disposed about the core of the first population of encapsulated particles is silica,
E. forming a second oil phase comprising the organohydrogensiloxane, and (d) second organopolysiloxane,
F. repeating steps B-D to form the second population of encapsulated particles disposed about the core of the second population of encapsulated particles is silica, and
G. combining the first and second populations of the encapsulated particles to form the dispersion.

One or more cationic surfactants, amphoteric surfactants, non-ionic surfactants, and/or other additives may be utilized.

The active ingredient may be in water dispersion or it can be enclosed into microcapsules as well. When encapsulated, the active drug can have a long lasting effect as it diffuses through the film towards the skin or other substrate. It can be encapsulated separately from compounds X and Y or it can be encapsulated with one of compounds X or Y and/or with the catalyst. The active ingredient can be encapsulated together with an excipient so that the drug is solubilised in a solvent which plays the role of skin penetration enhancer so as to optimize delivery of the drug.

### Additional Components

In addition to the components of the composition described above, the cores of any one or more of the encapsulated particles of the first and/or second populations, and/or the dispersion itself, may include one or more additional components. Similarly, the film itself may include one or more additional components to supplement those described above. These additional components may be silicone or organic components. In one embodiment, these components are substantially soluble with oil and substantially insoluble in water. Non-limiting examples of suitable additional components include silicones, such as volatile siloxanes, polydimethylsiloxane fluids, high molecular weight (i.e. molecular weight>1000) siloxanes, including silicone elastomers and resins, organic compounds such as, hydrocarbon oils, waxes, emollients, surfactants, thickeners, preservatives, antimicrobial, fragrances, colorants, coloured indicators, diluents, extenders, excipients, pH buffers, stabilizers, preservatives, surfactants, fluorinated silicones, processing aids such as cyclic or linear polydiorganosiloxanes, bioadhesive materials, and/or hydrophilic, modulating and swellable components or polymers. The delivery system preferably comprise an excipient, which is a pharmacologically inactive substance used as carrier for the active agent of the medication. Other non-limiting examples include absorbents for wounds, alginate, polysaccharides, gelatin, collagen, and materials that can decrease friction. Still other non-limiting examples include absorbents, anticaking agents, antioxidants, antistatic agents, astringents, binders, buffering agents, bulking agents, chelating agents, astringents, deodorants, emollients, film formers, flavouring agents, humectants, lytic agents, moisturizing agents, occlusivity enhancers, opacifying agents, oxidizing and reducing agents, penetration enhancers, plasticizers, preservatives, bleaching agents, conditioning agents, protectants, slip modifiers, solubilising agents, solvents, sunscreens, surface modifiers, surfactants and emulsifying agents, suspending agents, thickening agents, viscosity controlling agents, and UV light absorbers. Additional non-limiting examples of suitable additional components include alcohols, fatty alcohols and polyols, aldehydes, alkanolamines, alkoxylated alcohols (e.g. polyethylene glygol derivatives of alcohols and fatty alcohols), alkoxylated amides, alkoxylated amines, alkoxylated carboxylic acids, amides including salts (e.g. ceramides), amines, amino acids including salts and alkyl substituted derivatives, esters, alkyl substituted and acyl derivatives, polyacrylic acids, acrylamide copolymers, adipic acid copolymers, alcohols, aminosilicones, biological polymers and derivatives, butylene copolymers, carbohydrates (e.g. polysaccharides, chitosan and derivatives), carboxylic acids, carbomers, esters, ethers and polymeric ethers (e.g. PEG derivatives, PPG derivatives), glyceryl esters and derivatives, halogen compounds, heterocyclic compounds including salts, hydrophilic colloids and derivatives including salts and gums (e.g. cellulose derivatives, gelatin, xanthan gum, natural gums), imidazolines, inorganic materials (e.g. clay, TiO₂, ZnO), ketones (e.g. camphor), isethionates, lanolin and derivatives, organic salts, phenols including salts (e.g. parabens), phosphorus compounds (e.g. phosphate derivatives), polyacrylates and acrylate copolymers, protein and enzymes derivatives (e.g. collagen), synthetic polymers including salts, siloxanes and silanes, sorbitan derivatives, sterols, sulfonic acids and derivatives and waxes, salicylic acid, sulfur, calcium undecylenate, undecylenic acid, zinc undecylenate, povidone-iodine, alcohol, benzalkonium chloride, benzethonium chloride, hydrogen peroxide, methylbenzethonium chloride, phenol, poloxamer 188, acetyl cysteine, arbutin, ascorbic acid, ascorbic acid polypeptide, ascorbyl dipalmitate, ascorbyl methylsilanol pectinate, ascorbyl palmitate, ascorbyl stearate, BHA, p-hydroxyanisole, BHT, t-butyl hydroquinone, caffeic acid, camellia sinensis oil, chitosan ascorbate, chitosan glycolate, chitosan salicylate, chlorogenic acids, cysteine, cysteine HCl, decyl mercaptomethylimidazole, erythorbic acid, diamylhydroquinone, di-t-butylhydroquinone, dicetyl thiodipropionate, dicyclopentadiene/t-butylcresol copolymer, digalloyl trioleate, dilauryl thiodipropionate, dimyristyl thiodipropionate, dioleyl tocopheryl methylsilanol, isoquercitrin, diosmine, disodium ascorbyl sulfate, disodium rutinyl disulfate, di stearyl thiodipropionate, ditridecyl thiodipropionate, dodecyl gallate, ethyl ferulate, ferulic acid, hydroquinone, hydroxylamine hci, hydroxylamine sulfate, Isooctyl thioglycolate, kojic acid, madecassicoside, magnesium ascorbate, magnesium ascorbyl phosphate, melatonin, methoxy-PEG-7 rutinyl succinate, methylene di-t-butylcresol, methylsilanol ascorbate, nordihydroguaiaretic acid, octyl gallate, phenylthioglycolic acid, phloroglucinol, potassium ascorbyl tocopheryl phosphate, thiodiglycolamide, potassium sulfite, propyl gallate, rosmarinic acid, rutin, sodium ascorbate, sodium ascorbyl/cholesteryl, phosphate, sodium bisulfite, sodium erythorbate, sodium metabisulfide, sodium sulfite, sodium thioglycolate, sorbityl furfural, tea tree (melaleuca afiemifolia) oil, tocopheryl acetate, tetrahexyldecyl ascorbate, tetrahydrodiferuloylmethane, tocopheryl linoleate/oleate, thiodiglycol, tocopheryl succinate, thiodiglycolic acid, thioglycolic acid, thiolactic acid, thiosalicylic acid, thiotaurine, retinol, tocophereth-5, tocophereth-10, tocophereth-12, tocophereth-18, tocophereth-50, tocopherol, tocophersolan, tocopheryl linoleate, tocopheryl nicotinate, tocoquinone, o-tolyl biguanide, tris(nonylphenyl) phosphite, ubiquinone, and zinc dibutyldithiocarbamate, aluminium phenolsulfonate, ammonium phenolsulfonate, bakuchiol, benzalkonium bromide, benzalkonium cetyl phosphate, benzalkonium chloride, benzalkonium saccharinate, benzethonium chloride, potassium phenoxide, benzoxiquine, benzoxonium chloride, bispyrithione, boric acid, bromochlorophene, camphor benzalkonium methosulfate, captan, cetalkonium chloride, cetearalkonium bromide, cetethyldimonium bromide, cetrimonium bromide, cetrimonium chloride, cetrimonium methosulfate, cetrimonium saccharinate, cetrimonium tosylate, cetylpyridinium chloride, chloramine t, chlorhexidine, chlorhexidine diacetate, chlorhexidine digluconate, chlorhexidine dihydrochloride, p-chloro-m-cresol, chlorophene, p-chlorophenol, chlorothyrnol, chloroxylenol, chlorphenesin, ciclopirox olamine, climbazole, cloflucarban, clotrimazole, coal tar, colloidal sulfur, o-cymen-5-ol, dequalinium acetate, dequalinium chloride, dibromopropamidine diisethionate, dichlorobenzyl alcohol, dichlorophene, dichlorophenyl imidazoldioxolan, dichloro-m-xylenol, diiodomethyltolylsulfone, dimethylol ethylene thiourea, diphenylmethyl piperazinylbenzimidazole, domiphen bromide, 7-ethylbicyclooxazolidine, fluorosalan, formaldehyde, glutaral, hexachlorophene, hexamidine, hexamidine diisethionate, hexamidine diparaben, hexamidine paraben, hexetidine, hydrogen peroxide, hydroxymethyl dioxoazabicyclooctane, ichthammol, isopropyl cresol, lapyrium chloride, lauralkonium bromide, lauralkonium chloride, laurtrimonium bromide, laurtrimonium chloride, laurtrimonium trichlorophenoxide, lauryl isoquinolinium bromide, lauryl isoquinolinium saccharinate, laurylpyridinium chloride, mercuric oxide, methenamine, methenammonium chloride, methylbenzethonium chloride, myristalkonium chloride, myristalkonium saccharinate, myrtrimonium bromide, nonoxynol-9 iodine, nonoxynol-12 iodine, olealkonium chloride, oxyquinoline, oxyquinoline benzoate, oxyquinoline sulfate, PEG-2 coco-benzonium chloride, PEG-10 coco-benzonium chloride, PEG-6 undecylenate, PEG-8 undecylenate, phenol, o-phenylphenol, phenyl salicylate, piroctone olamine, sulfosuccinylundecylenate, potassium o-phenylphenate, potassium salicylate, potassium troclosene, propionic acid, pvp-iodine, quaternium-8, quaternium-14, quaternium-24, sodium phenolsulfonate, sodium phenoxide, sodium o-phenylphenate, sodium shale oil sulfonate, sodium usnate, thiabendazole, 2,2′-thiobis(4-chlorophenol), thiram, triacetin, triclocarban, triclosan, trioctyldodecyl borate, undecylenamidopropylamine oxide, undecyleneth-6, undecylenic acid, zinc acetate, zinc 30 aspartate, zinc borate, zinc chloride, zinc citrate, zinc cysteinate, zinc dibutyldithiocarbamate, zinc gluconate, zinc glutamate, zinc lactate, zinc phenolsulfonate, zinc pyrithione, zinc sulfate, and zinc undecylenate, benzyl alcohol, capsicum oleoresin (capsicum frutescens oleoresin), methyl salicylate, camphor, phenol, capsaicin, juniper tar (juniperus oxycedrus tar), phenolate sodium (sodium phenoxide), capsicum (capsicum frutescens), menthol, resorcinol, methyl nicotinate, and turpentine oil (turpentine), ammonium persulfate, calcium peroxide, hydrogen peroxide, magnesium peroxide, melamine peroxide, potassium bromate, potassium caroate, potassium chlorate, potassium persulfate, sodium bromate, sodium carbonate peroxide, sodium chlorate, sodium iodate, sodium perborate, sodium persulfate, strontium dioxide, strontium peroxide, urea peroxide, zinc peroxide, ammonium bisufite, ammonium sulfite, ammonium thioglycolate, ammonium thiolactate, cystemaine HCl, cystein, cysteine HCl, ethanolamine thioglycolate, glutathione, glyceryl thioglycolate, glyceryl thioproprionate, hydroquinone, p-hydroxyanisole, isooctyl thioglycolate, magnesium thioglycolate, mercaptopropionic acid, potassium metabisulfite, potassium sulfite, potassium thioglycolate, sodium bisulfite, sodium hydrosulfite, sodium hydroxymethane sulfonate, sodium metabisulfite, sodium sulfite, sodium thioglycolate, strontium thioglycolate, superoxide dismutase, thioglycerin, thioglycolic acid, thiolactic acid, thiosalicylic acid, zinc formaldehyde sulfoxylate, hydroquinone, allantoin, aluminium acetate, aluminium hydroxide, aluminium sulfate, calamine, cocoa butter, cod liver oil, colloidal oatmeal, dimethicone, glycerin, kaolin, lanolin, mineral oil, petrolatum, shark liver oil, sodium bicarbonate, talc, witch hazel, zinc acetate, zinc carbonate, zinc oxide, aminobenzoic acid, cinoxate, diethanolamine methoxycinnamate, digalloyl trioleate, dioxybenzone, ethyl 4-[bis(hydroxypropyl)] aminobenzoate, glyceryl aminobenzoate, homosalate, lawsone with dihydroxyacetone, menthyl anthranilate, octocrylene, octyl methoxycinnamate, octyl salicylate, oxybenzone, padimate 0, phenylbenzimidazole sulfonic acid, red petrolatum, sulisobenzone, titanium dioxide, trolamine salicylate, acetaminosalol, allatoin PABA, benzalphthalide, benzophenone, benzophenone 1-12, 3-benzylidene camphor, benzylidenecamphor hydrolyzed collagen sulfonamide, benzylidene camphor sulfonic acid, benzyl salicylate, bornelone, bumetriozole, butyl methoxydibenzoylmethane, butyl PABA, ceria/silica, ceria/silica talc, cinoxate, DEA-methoxycinnamate, dibenzoxazol naphthalene, di-t-butyl hydroxybenzylidene camphor, digalloyl trioleate, diisopropyl methyl cinnamate, dimethyl PABA ethyl cetearyldimonium tosylate, dioctyl butamido triazone, diphenyl carbomethoxy acetoxy naphthopyran, disodium bisethylphenyl tiamminotriazine stilbenedisulfonate, disodium distyrylbiphenyl triaminotriazine stilbenedisulfonate, disodium distyrylbiphenyl disulfonate, drometrizole, drometrizole trisiloxane, ethyl dihydroxypropyl PABA, ethyl diisopropylcinnamate, ethyl methoxycinnamate, ethyl PABA, ethyl urocanate, etrocrylene ferulic acid, glyceryl octanoate dimethoxycinnamate, glyceryl PABA, glycol salicylate, homosalate, isoamyl pmethoxycinnamate, isopropylbenzyl salicylate, isopropyl dibenzolylmethane, isopropyl methoxycinnamate, menthyl anthranilate, menthyl salicylate, 4-methylbenzylidene, camphor, octocrylene, octrizole, octyl dimethyl PABA, octyl methoxycinnamate, octyl salicylate, octyl triazone, PABA, PEG-25 PABA, pentyl dimethyl PABA, phenylbenzimidazole sulfonic acid, polyacrylamidomethyl benzylidene camphor, potassium methoxycinnamate, potassium phenylbenzimidazole sulfonate, red petrolatum, sodium phenylbenzimidazole sulfonate, sodium urocanate, tea-phenylbenzimidazole sulfonate, tea-salicylate, terephthalylidene dicamphor sulfonic acid, titanium dioxide, tripaba panthenol, urocanic acid, and VA/crotonates/methacryloxybenzophenone-1 copolymers. To the extent that one or more of the aforementioned compounds may also be a pharmaceutically active agent, such as a drug, therapeutic agent, etc., it is contemplated that such a compound could alternatively be utilized as the active agent in this disclosure.

Preferably, the microcapsules have a size such that the average particle size according to D(v,0.5) is less than 30 micrometers, more preferably less than 20 micrometer. This is especially appropriate to ensure that upon application, the microcapsules burst and release their content to allow hydrosilylation reaction to form an adhesive silicone film.

The invention relates to water-based, drug containing, hydrosilylation reactive microcapsule compositions having extended bath life and superior waterproofing and their application in dermal or transdermal drug delivery.

### EXAMPLES

### Raw Materials

| | Typical | | |
|---|---|---|---|
| | Concentration | | Abbreviated |
| Chemical Name | (%) | Function | Name |
| Dimethyl Siloxane, | 96-98 | Polymer | Vinyl siloxane |
| Dimethylvinylsiloxy- | | | |
| terminated | | | |
| Viscosity = 300-600 | | | |
| mPa · s (cPs) at 25° C. | | | |
| Vinyl functionalisation: | | | |
| 0.448% (w/w) | | | |
| Karstedt's catalyst 1,3- | 2-4 | Catalyst | Pt catalyst |
| Diethenyl-1,1,3,3-Tetra- | | | |
| methyldisiloxane | | | |
| Complexes containing 520 | | | |
| ppm [Pt IV] | | | |

| | Typical | | |
|---|---|---|---|
| | Concentration | | Abbreviated |
| Chemical Name | (%) | Function | Name |
| Dimethyl Siloxane, | 50-93.7 | Polymer | Vinyl siloxane |
| Dimethylvinylsiloxy- | | | |
| terminated | | | |
| Viscosity = 300-600 | | | |
| cPs and 0.448% Vinyl | | | |
| functionalized | | | |
| Dimethyl, Methyl- | 6.25-50 | Crosslinker | SiH Polymer |
| hydrogen siloxane, | | | |
| Trimethylsiloxy- | | | |
| terminated Viscosity = 5 | | | |
| cPs and 0.795% SiH | | | |
| functionalised | | | |

The viscosity of the vinyl siloxane and SiH siloxane was measured at 25° C. according to the Dow Corning CTM 0050 method using a Brookfield rotating viscosimeter with a RVF2 spindle at 20 rpm.

### Example 1

On one hand, 0.68 g of cetyl trimethyl ammonium chloride (CTAC) as emulsifier is added to 158.37 g of water under mild stirring until clear. A blend, consisting in 135.12 g of vinylsiloxane and 4.2 g of Pt catalyst, is added under mixing at 400 rpm to the CTAC/Water mixture in order to make an O/W emulsion by the means known by the man of the art to make a coarse O/W emulsions. In this particular case an Ultra-Turrax T25 Basic has been utilised for 90 seconds at 9500 rpm. A finer O/W is made by using an APV 1000 Homogeniser, or similar technique known by the man of the art to make fine emulsions or microemulsions, at a pressure of 700 bars in order to obtain droplets having Dv 0.9 below 15 μm.

pH is set to 3.7 by addition of HCl then 12.86% of tetraethylorthosilicate (TEOS) is added under mixing at 400 rpm for 4 hours. After complete hydrolysis and condensation of the TEOS, a suspension of Core-Shell microcapsules of average volume particle size (Dv 0.5)=2.2 micrometers was produced. The suspension was then diluted down with water in order to obtain a solid content of 30%.

Optionally 0.3% of 3-(Trimethoxysilyl)propyldimethylhexadecylammonium Chloride is then added to the suspension to prevent gelation at 45° C.

On the other hand, 0.68 g of cetyl trimethyl ammonium chloride (CTAC) was added to 162.69 g of water under mild stirring until clear. The base blend, consisting in 120 g vinyl siloxane and 15 g of SiH Polymer, was added under mixing at 400 rpm to the CTAC/Water mixture in order to make an O/W emulsion by the means known by the man of the art to make a coarse O/W emulsions. In this particular case an Ultra-Turrax T25 Basic has been utilised for 90 seconds at 9500 rpm. A finer O/W is made by using an APV 1000 Homogeniser, or any other technique known by the man of the art to make fine emulsions or microemulsions, at a pressure of 700 bars in order to obtain droplets having Dv 0.9 below 15 μm.

pH was set to 3.7 by addition of HCl then 12.86% of tetraethylorthosilicate (TEOS) was added under mixing at 400 rpm for 4 hours. After complete hydrolysis and condensation of the TEOS, a suspension of Core-Shell microcapsules of average volume particle size (Dv 0.5)=1.73 micrometers was produced. The suspension was then diluted down with water in order to obtain a solid content of 30%.

0.3% of 3-(Trimethoxysilyl)propyldimethylhexadecylammonium Chloride was then added to the suspension to prevent gelation at 45° C.

Microcapsules of the catalyst and base blends were then mixed together at a 1/1 w/w.

A drug or a mix of drugs is added to the suspension blend and conditioned into a pump spray and applied onto the skin.

Alternatively the microcapsules suspension blend containing at least one drug is conditioned into a high pressure aerosol or a roller.

Particle size measurements were made by laser diffraction technique using a “Mastersizer 2000” from Malvern Instruments Ltd., UK. (Further information on the particle size determination can be found in “Basic Principles of Particle Size Analytics”, Dr. Alan Rawle, Malvern Instruments Limited, WR14 1XZ, UK and the “Manual of Malvern Particle Size Analyser”. Particular reference is made to the user manual number MNA 0096, Issue 1.0, November 1994. All particle sizes indicated in the present application are mean average particle size according to D(v,0.5) and are measured with a Malvern Mastersizer.

1. A method for the therapeutic treatment of a keratinous substrate or mucous membrane comprising preparing a composition containing water, at least one therapeutically active ingredient, an organopolysiloxane X having at least two silicon-bonded alkenyl groups per molecule, a hydrosilylation catalyst, and an organohydrogensiloxane compound Y having at least two silicon bonded hydrogen atoms per molecule, wherein at least one of compound X or Y is enclosed within microcapsules suspended in an aqueous phase, and applying the composition to a keratinous substrate or mucous membrane.

2. The method according to claim 1, further comprising applying said composition in galenic form so that a hydrosilylation reaction between compounds X and Y occurs to form a film on the keratinous substrate or mucous membrane.

3. The method according to claim 2, wherein the galenic form is a spray, foam, brush, pen or roller.

4. The method according to claim 1, wherein compounds X and Y are present in separate encapsulated forms.

5. The method according to claim 2, wherein a first portion of the microcapsules comprise the compound X and the catalyst and a second portion comprises the compound Y, optionally associated with the compound X.

6. The method according to claim 1, wherein the molar portion of SiH/unsaturated groups ranges from 1 to 20.

7. The method according to claim 1, wherein said organopolysiloxane X comprises at least two siloxane units per molecule that each independently have the average formula R2RmSiO(3-m)/2, wherein each R is independently a hydrocarbon group having from 1 to 20 carbon atoms, each R2 is a monovalent alkenyl aliphatic group, and m is a number of from 0 to 2.

8. The method according to claim 1, wherein said organopolysiloxane X comprises units of average formula R2R2SiO1/2, R2RSiO and/or R2SiO3/2 wherein each R is independently a hydrocarbon group having from 1 to 20 carbon atoms and each R2 is a monovalent alkenyl aliphatic group.

9. The method according to claim 1, wherein said organopolysiloxane X has an average formula that is defined as:
CH₂═CH(Me)₂SiO[Me₂SiO]_{x′}Si(Me)₂CH═CH₂;
CH₂═CH—(CH₂)₄—(Me)₂SiO[Me₂SiO]_{x′}Si(Me)₂—(CH₂)₄—CH═CH_{2;}
or
Me₃SiO[(Me)₂SiO]_{x′}[CH₂═CH(Me)SiO]_{x″}SiMe₃,
and wherein Me is methyl, x′≧0, and x″≧2.

10. The method according to claim 8, wherein said organohydrogensiloxane compound Y has an average formula
(R33SiO1/2)a(R42SiO2/2)b(R4HSiO2/2)c, wherein:
each R3 is independently a hydrogen atom or R4,
each R4 is independently a monovalent hydrocarbyl having 1 to 10 carbon atoms, and
wherein a≧2, b≧0, and c≧2.

11. The method according to claim 9, wherein said organohydrogensiloxane Y is a dimethyl, methyl-hydrogen polysiloxane having an average formula (CH3)3SiO[(CH3)2SiO]b[(CH3)HSiO]cSi(CH3)3, wherein b≧0, and c≧2.

12. The method according to claim 1, wherein the hydrosilylation catalyst is a platinum group metal present at a concentration of 1 to 500 parts per million relative to the total weight of film.

13. The method according to claim 1, wherein the composition contains from 0.001 to 20 weight percent therapeutically active ingredient based on the total weight of the dispersion.

14. The method according to claim 1, wherein the microcapsule shell comprises silica.

15. The method according to claim 1, wherein the shell of the microcapsules are formed from precursors comprising tetraalkoxysilane.

16. The method according to claim 1, wherein the microcapsules have a mean average particle size according to D(v,0.5) of less than 20 micrometers.

17. (canceled)

18. (canceled)

19. (canceled)

20. The method according to claim 1, wherein the therapeutically active ingredient is selected from antibacterials, antibiotics, antimicrobials, corticosteroids, analgesics, and anti-inflammatory agents.

21. The method according to claim 1 wherein the therapeutically active ingredient is hydorcortisone, clobetasol propionate, betamethasone, ibuprofen, aspirin, diclofenac (and salts thereof), lidocaine, salicylic acid, or clidamycin phosphate.
